(19) United States
(12) Patent Application Publication
(10) Pub. No.: US 2006/0148362 A1
(43) Pub. Date: Jul. 6, 2006

(54) Disposable nonwoven undergarments with support panels

(76) Inventor: Cliff Bridges, Greensboro, NC (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 11/287,785

(22) Filed: Nov. 28, 2005

Related U.S. Application Data
(60) Provisional application No. 60/631,724, filed on Nov. 30, 2004.

(51) Int. Cl. A61F 13/15; D04H 1/46; D04H 1/00

(52) U.S. Cl. 442/408; 604/387; 442/415; 442/417

(57) ABSTRACT

The present invention is directed to a soft feeling, aesthetically pleasing, and form-fitting disposable nonwoven undergarment for men, women, and youths with one or more expandable supportive body panels, as well as an optional absorbent protective panel for those individuals with mild incontinence conditions. In accordance with the present invention, the form-fitting disposable nonwoven undergarment is comprised of one or more expandable body panels for those individuals that require or prefer additional support in an undergarment.

### CROSS-REFERENCE TO RELATED APPLICATION

This application claims the benefit of priority Provisional Application No. 60/631,724 filed Nov. 30, 2004, the disclosures of which is incorporated herein by reference.

### TECHNICAL BACKGROUND

The present invention relates to a soft feeling, aesthetically pleasing, and form-fitting disposable nonwoven undergarment for men, women, and youths, and more specifically relates to form-fitting disposable undergarments with one or more expandable support panels, as well as an optional absorbent protective panel for those individuals with mild incontinence conditions.

### BACKGROUND OF THE INVENTION

Undergarments are those garments that come into direct contact with the skin. They are usually worn under an outer layer of clothing and remain generally unseen by the public. Undergarments cover a variety of constructs such as basic, woven, cotton undershirts and underpants for daily wear, absorbent disposable undergarments such as diapers and incontinence devices used to absorb and retain liquid waste, as well as specialty undergarments such as travel wear, medical wear, and athletic wear.

Disposable undergarments are widely accepted due to the convenience of use, however, such undergarments are often cumbersome, uncomfortable, noisy, and aesthetically unappealing. Prior art discloses disposable undergarments for incontinence purposes, traveling convenience, fem-care products, and medical wear.

Glaug, U.S. Pat. No. 6,307,120 discloses an absorbent incontinence article whereby the absorbent incontinence article is a cloth-like, breathable, disposable, brief comprising plural fastening tapes.

Fell, et al, H1, 969 discloses an absorbent disposable undergarment that utilizes a breathable micro-porous film as a backing member to improve absorbent and containment characteristics, as well as, to improve comfort for garments such as incontinence devices.

Meisels, U.S. Pat. No. 5,103,501 discloses three articles of disposable underwear for use during travel. The undershirt, men's underpants, and women's underpants, contain magnetic fasteners in order to minimize the amount of dressing time necessary with use of these garments and prevent tearing of the garment during removal.

Greene, U.S. Pat. No. 4,674,135 discloses a durable disposable undergarment that is made of nonwoven cellulosic paper or paper-like material, in which the garment is intended for use by travelers. The disposable garment includes a small bag to be used upon disposal of the garment.

Rajala, et al., U.S. Pat. No. 6,260,211 discloses a disposable undergarment that provides back-up protection for women in the event that a sanitary napkin should leak.

Murakami, et al., U.S. Pat. No. 6,289,519 discloses a disposable, “trunk-type”, undergarment constructed of a spunbond nonwoven, whereby the trunks are intended for use by medical team members such as doctors and nurses, as well as patients.

The prior art lacks a durable, disposable, nonwoven undergarment that offers the comfort of supportive body panels and that is suitable for daily use. The majority of disposable undergarments are directed toward diapers and incontinence devices or fail to provide the comfort necessary for daily use. Further, for those individuals suffering from a mild incontinence condition, the only available undergarment suitable for use are those of a “diaper-like” construction, which exhibit a negative aesthetic appeal. In addition, available incontinence devices are designed to withstand a high volume of liquid, which makes the traditional, “diaper-like”, incontinence device too burdensome. Previously mentioned disposable undergarments are either noisy or contain unpleasant fasteners.

It is the object of the present invention to provide a soft feeling, aesthetically pleasing, and form-fitting disposable undergarment with one or more supportive body panels that offers the wearer reassuring comfort. The present invention is durable and intended for daily use, but is also useful in disposable athletic, travel, or medical undergarment, and when so constructed, as a disposable undergarment for mild incontinence conditions.

### SUMMARY OF THE INVENTION

The present invention is directed to a soft feeling, aesthetically pleasing, and form-fitting disposable nonwoven undergarment for men, women, and youths with one or more expandable supportive body panels, as well as an optional absorbent protective panel for those individuals with mild incontinence conditions.

In accordance with the present invention, the form-fitting disposable nonwoven undergarment is comprised of one or more expandable body panels for those individuals that require or prefer additional support in an undergarment. Supportive undergarments are often suggested to those individuals that undergo certain surgical procedures, such as a hernia repair for example. Still a percentage of the population prefers supportive undergarments for aesthetic or cosmetic reasons.

The supportive undergarment of present invention utilizes nonwoven fabrics that exhibit suitable hand, drapeability, and a three-dimensional image or pattern to provide for a comfortably fitting, fashionable, disposable supportive undergarment. The present invention discloses an assortment of durable, disposable supportive undergarments capable of meeting the needs of the general public, as well as specific subsets of the population such as travelers, athletes, medical personnel and patients, for which routine care of conventional undergarments is inconvenient or unavailable.

The supportive undergarment is comprised of at least one supportive panel that exhibits a controlled expandable performance. Suitable substrates which may be incorporated into the undergarment as one or more supportive panels include, but not limited to those nonwoven fabrics exemplified in U.S. Pat. No. 6,617,016; No. 6,506,698, No. 5,865,926; and No. 5,681,645, all of which are hereby incorporated by reference. A preferred supportive panel substrate is breathable, cloth-like and comfortable against the skin, and provides a controlled expandability to lend a supportive characteristic to the undergarment.

It is also in the purview of the present invention to incorporate an absorbent protective panel for individuals with a mild incontinence condition, who require a device suitable for limited urinary containment. Further, the supportive undergarment may comprise one or more medicinal agents in one or more regions of the undergarment to treat those ailments that affect the lower torso of the body, including the hips, buttock, and groin area.

Other features and advantages of the present invention will become readily apparent from the following detailed description, the accompanying drawings, and the appended claims.

### BRIEF DESCRIPTION OF THE DRAWINGS

FIG. 1 is a depiction of the apparatus for the fabrication of the nonwoven fabric according to the present invention;

FIG. 2 is a perspective view of a male “brief” style disposable undergarment according to the present invention;

FIG. 3 is a perspective view of a male “bikini” style disposable undergarment according to the present invention;

FIG. 4 is a perspective view of a female “brief” style disposable undergarment according to the present invention;

FIG. 5 is a perspective view of a female “bikini” style disposable undergarment according to the present invention; and

FIGS. 6 and 7 are a perspective views of a male sport “brief” style disposable undergarment according to the present invention.

### DETAILED DESCRIPTION OF THE INVENTION

While the present invention is susceptible of embodiment in various forms, hereinafter is described presently preferred embodiments of the invention, with the understanding that the present disclosure is to be considered as exemplifications of the invention, and is not intended to limit the invention to the specific embodiments illustrated.

The nonwoven fabric of the disposable supportive undergarment of the present invention is formed from natural fibers, synthetic fibers, or a combination of natural and synthetic fibers. The fibers may be of finite staple length, continuous filaments and the blends thereof. Synthetic fibers may be selected from thermoset polymers such as polyacrylates, or from thermoplastic polymers, including; polyamides, polyesters, or polyolefins, such as polypropylene or polyethylene, their derivatives, and combinations thereof. The synthetic fibers of the present invention may also include any fibers with multi-component configurations, such as side-by-side or sheath-core, as well as geometric variations. The natural fibers of the present invention are cellulosic in nature such as cotton, wood pulp, or rayon.

The nonwoven fabric used in accordance with the present invention should be durable and able to withstand the stress of continual body movement involved with everyday activities. The fabric should also have suitable drape and hand properties so that the fabric will conform to the curves of the body. In addition, the fabric should have a hydrophilic and hydrophobic sides in order absorb and repel moisture where needed.

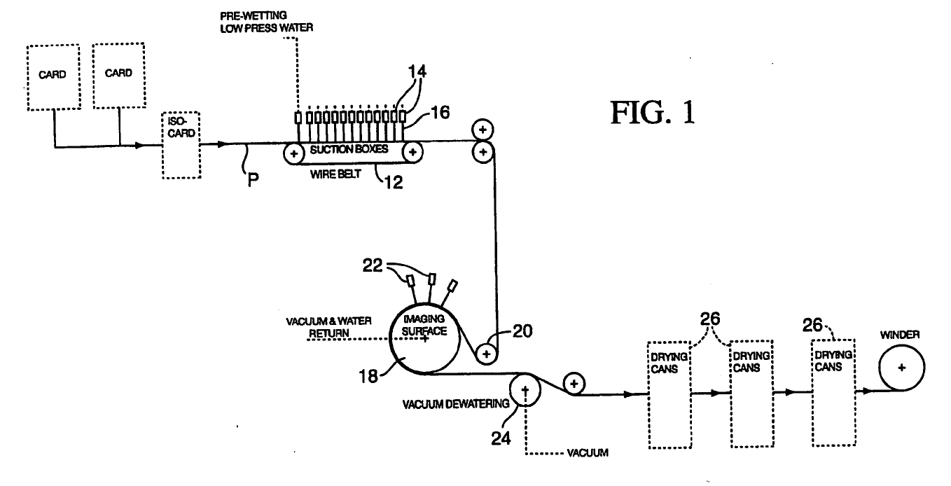

In reference to FIG. 1, therein is illustrated an apparatus for practicing the method of the present invention for forming a nonwoven fabric. The fabric is formed from a fibrous matrix, which comprises fibers selected to promote economical manufacture, while achieving the desired resultant nonwoven fabric. The fibrous matrix is preferably carded and subsequently air-randomized to form a precursor web, designated P.

FIG. 1 illustrates a hydroentangling apparatus for forming nonwoven fabrics in accordance with the present invention. The apparatus includes a foraminous forming surface in the form of a flat bed entangler 12 upon which the precursor web P is positioned for pre-entangling. Precursor web P is then sequentially passed under entangling manifolds 14, whereby the precursor web is subjected to high-pressure water jets 16. This process is well known to those skilled in the art and is generally taught by U.S. Pat. No. 3,485,706, to Evans, hereby incorporated by reference.

The entangling apparatus of FIG. 1 further includes an imaging and patterning drum 18 comprising a three-dimensional image transfer device for effecting imaging and patterning of the now-entangled precursor web. After pre-entangling, the precursor web is trained over a guide roller 20 and directed to the image transfer device 18, where a three-dimensional image is imparted into the fabric on the foraminous forming surface of the device. The web of fibers is juxtaposed to the image transfer device 18, and high pressure water from manifolds 22 is directed against the outwardly facing surface from jet spaced radially outwardly of the image transfer device 18. The image transfer device 18, and manifolds 22, may be formed and operated in accordance with the teachings of commonly assigned U.S. Pat. No. 5,098,764, No. 5,244,711, No. 5,822,823, and No. 5,827,597, the disclosures of which are hereby incorporated by reference. It is presently preferred that the precursor web P be given a three-dimensional image suitable to provide fluid management, as will be further described, to promote use of the present nonwoven fabric in disposable absorbent articles. The entangled fabric can be vacuum dewatered at 24, and dries at an elevated temperature on drying cans 26.

The nonwoven fabric of the present invention may be a composite, laminate, single layer or multiple layers in order to incorporate support and/or absorbent mechanisms into the undergarment. The nonwoven may be imaged, such as with ribs or swirls, etc., apertured, or modified aesthetically through subsequent dyeing, and printing, or by using colored fibers during the manufacturing step, to achieve the effects of the desired nonwoven disposable undergarment. The nonwoven fabric has a preferred basis weight range of 1.5-5.5 ounces per square yard, with a range of 2.0-3.0 ounces per square yard being most preferred.

A disposable nonwoven undergarment has skin health and hygiene benefits. The breathable and moisture absorbing characteristics of the nonwoven fabric utilized in the disposable undergarment provides the skin with a comfortable environment. The nonwoven fabric helps keep the skin oxygenated and dry, which is beneficial for those individuals with an active lifestyle. The disposable undergarment is also equipped with one or more supportive panels for additional comfort. Exemplary fabric that may be suitable to incorporate as supportive panels are taught in U.S. Pat. No. 6,617,016; No. 6,506,698, No. 5,865,926; and No. 5,681,645, previously incorporated herein by reference.

The nonwoven disposable undergarment is typically of the representative “brief” design, wherein the undergarment is specific to gender and end use application. The brief of the present invention may be scaled to various sizes, i.e. small, medium, large, etc, in order to accommodate various body types on forms. In general, the undergarment is comprised of one or more pieces that are preferentially thermally welded, mechanically stitched, or adhesively bonded at the seams. Each undergarment comprises a waist opening at the upper portion of the undergarment and a pair of leg openings at the lower portion of the undergarment. The undergarment may optionally contain an elastic portion around the waist and leg openings.

In one embodiment, the disposable undergarment comprises a front panel, back panel, and interconnecting crotch region, wherein said front, back panel, and an interconnecting crotch region are mechanically secured together at their transverse edges. The nonwoven undergarment contains two elasticized leg openings and an elasticized waist. The imaged nonwoven undergarment consists of 50% polyester and 50% rayon, whereby the nonwoven fabric is of a layered construct, wherein the soft, hydrophilic rayon fabric layer is inside the undergarment against the skin while the hydrophobic, polyester fabric makes up the facing of the undergarment. The disposable undergarment of this embodiment also contains a discrete cellulosic protective panel imparted to the interconnecting crotch region for moisture management.

The disposable undergarment further comprises one or more supportive panels positioned within the aforementioned front and/or back panels of the undergarment. Preferably, the supportive panels comprise one or more breathable nonwoven fabrics that demonstrate an extensible performance, which may offer more or less support as needed depending the construct and composition of the supportive panel. Suitable fabrics that can be used as a supportive panel include, but are not limited to films, staple fiber webs, discontinuous filament webs, continuous filament webs, and the combination thereof. In addition, one or more of the supportive panel fabrics may be imparted with raised portions or images that are similar or dissimilar to the images imparted into the disposable nonwoven undergarment front and back panels, and interconnecting crotch region.

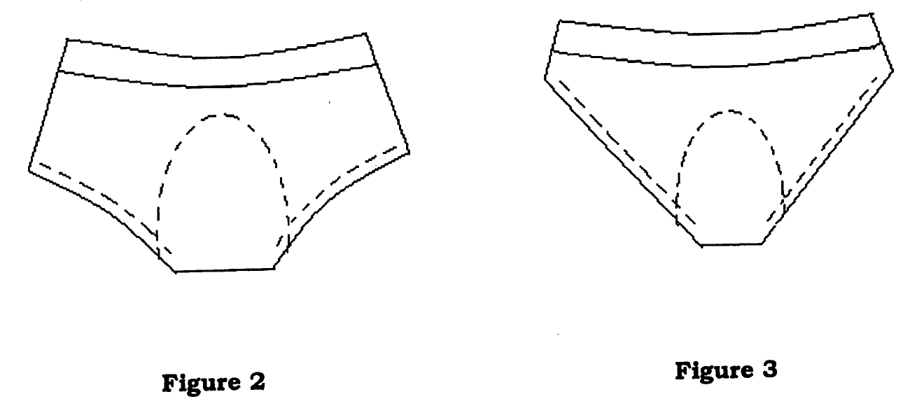

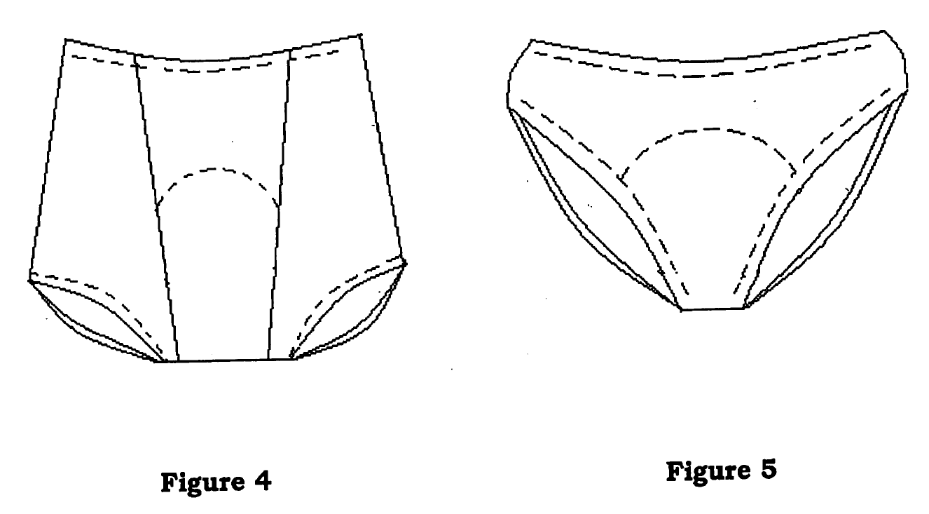

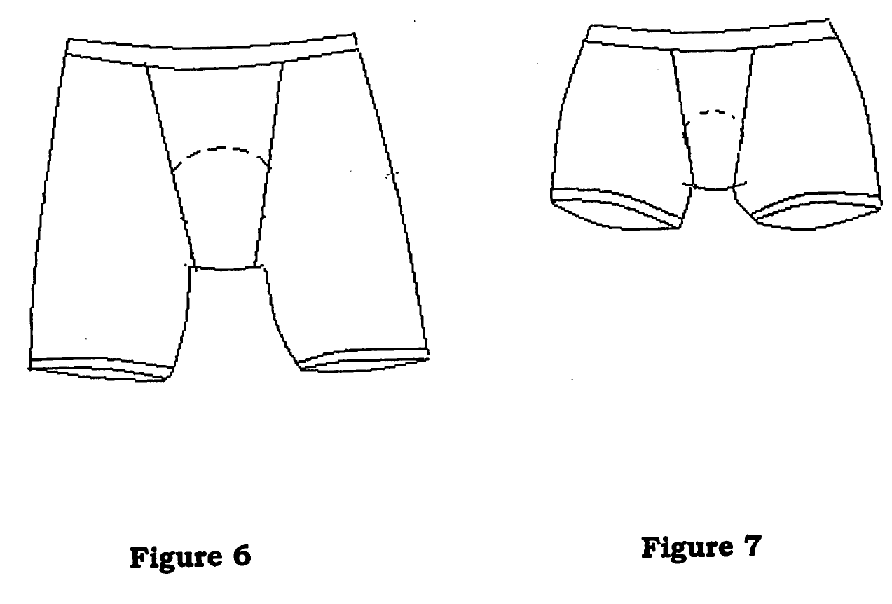

It has also been contemplated to incorporate more than one supportive panel within the same disposable undergarment, as in FIG. 6, wherein the first supportive panel offers a first extensibility performance and the second supportive panel offers a second extensibility performance. Further, a single supportive panel may comprise more than one zone of extensibility, as shown in FIG. 5. Further still, the one or more supportive panels may be incorporated in the form of a “pouch” or “pocket”, illustrated in FIG. 2, whereby the pouch may comprise anticipated, or perceived medicinal agents, such as antibiotics and anti-inflammatory pharmaceuticals, as well as holistic agents, such as essential oils, teas, and other aromatics. The medicinal agents may also be affixed or otherwise imparted to the inside of the disposable undergarment in the form of aqueous solutions, gels, waxes, or powders, wherein said medicinal agent is administered to the skin via normal contact, wearer motion, and/or body heat.

It is within the purview of the present invention to provide medical personnel and patients, male and female athletes, individuals that travel, and individuals with mild incontinence conditions with a comfortable, yet durable nonwoven disposable undergarment. Each disposable undergarment being tailored to the needs of these individuals, but all them having in common a soft feeling, aesthetically pleasing, form-fitting supportive quality that is currently unavailable in disposable undergarments.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed is:

1. A process for fabricating a disposable nonwoven undergarment comprising at least one support panel:
providing a precursor nonwoven web;
providing at least one supportive panel;
providing a three-dimensional image transfer device;
hydroentangling said precursor web on said image transfer device to form a patterned and imaged nonwoven fabric;
forming said patterned and imaged nonwoven fabric into a front panel, a back panel, and an interconnecting crotch region; and
joining said supportive panel, front panel, said back panel, and said interconnecting crotch region to form a disposable undergarment.

2. A process for fabricating a disposable nonwoven undergarment comprising at least one support panel as in claim 1, wherein said precursor web is a fibrous blend of synthetic and natural fibers.

3. A process for fabricating a disposable nonwoven undergarment comprising at least one support panel as in claim 2, wherein said synthetic fibers are selected from thermoset polymers such as polyacrylates or thermoplastic polymers, including; polyamides, polyesters, or polyolefins, such as polypropylene or polyethylene, their derivatives, and combinations thereof.

4. A process for fabricating a disposable nonwoven undergarment comprising at least one support panel as in claim 3, wherein said synthetic fibers are polyester.

5. A process for fabricating a disposable nonwoven undergarment comprising at least one support panel as in claim 2, wherein said natural fibers are cellulosic in nature such as cotton, wood pulp, or rayon.

6. A process for fabricating a disposable nonwoven undergarment comprising at least one support panel as in claim 5, wherein said natural fibers are rayon.

7. A process for fabricating a disposable nonwoven undergarment comprising at least one support panel as in claim 1, wherein said disposable nonwoven undergarment is imparted with a pattern or three-dimensional image, such as ribs, swirls, etc., by means of an image transfer device.

8. A process for fabricating a disposable nonwoven undergarment comprising at least one support panel as in claim 1, wherein said formed disposable nonwoven undergarment is comprised of one patterned or imaged nonwoven fabric piece.

9. A process for fabricating a disposable nonwoven undergarment comprising at least one support panel as in claim 7, wherein said patterned or imaged nonwoven fabric pieces are joined by thermally welded, mechanically stitched, or adhesively bonded.

10. A process for fabricating a disposable nonwoven undergarment comprising at least one support panel as in claim 8, wherein said pieces are mechanically stitched.

11. A process for fabricating a disposable nonwoven undergarment comprising at least one support panel as in claim 1, wherein said undergarment is for daily use, travelers, or athlete.

12. A process for fabricating a disposable nonwoven undergarment comprising at least one support panel as in claim 1, wherein said undergarment comprises a moisture absorbent panel for mild incontinence purposes.

13. A process for fabricating a disposable nonwoven undergarment comprising at least one support panel as in claim 7, wherein said formed disposable nonwoven undergarment piece comprises elastically lined leg openings and waist opening.

14. A process for fabricating a disposable nonwoven undergarment comprising at least one support panel as in claim 1, wherein said supportive panel is comprised of a breathable and extensible fabric.

15. A process for fabricating a disposable nonwoven undergarment comprising at least one support panel as in claim 14, wherein said supportive panel is comprised of more than zone of extensibility.

16. A process for fabricating a disposable nonwoven undergarment comprising at least one support panel as in claim 14, wherein said breathable and extensible fabric is a laminate.

17. A process for fabricating a disposable nonwoven undergarment comprising at least one support panel as in claim 1, wherein said supportive panel takes the form of a pouch.

18. A process for fabricating a disposable nonwoven undergarment comprising at least one support panel as in claim 1, wherein said undergarment comprises a medicinal agent.

19. A process for fabricating a disposable nonwoven undergarment comprising at least one support panel as in claim 18, wherein said medicinal agent is selected from the group consisting of pharmaceuticals and holistic agents.
